(19) United States
(12) Patent Application Publication
(10) Pub. No.: US 2023/0200718 A1
(43) Pub. Date: Jun. 29, 2023

(54) INFLAMMATORY RESPONSE TEST KIT

(71) Applicant: Micron Technology, Inc., Boise, ID (US)

(72) Inventors: Srinivasa Anuradha BULUSU, Boise, ID (US); Kathryn H. RUSSO, Boise, ID (US); Mandy W. FORTUNATI, Boise, ID (US)

(73) Assignee: Micron Technology, Inc., Boise, ID (US)

(21) Appl. No.: 17/561,091

(22) Filed: Dec. 23, 2021

(51) Int. Cl. A61B 5/00; A61B 5/145

(52) U.S. Cl. CPC A61B 5/411; A61B 5/14517; A61B 5/7475; A61B 5/0002; A61B 5/0077

(57) ABSTRACT

An allergy test kit including a housing, a biosensor supported by the housing, the biosensor operable to detect a presence of immunoglobin A (IgA) in a sample of sweat of a user and to generate IgA presence data, an allergen sensor supported by the housing, the allergen sensor operable to detect a presence of an allergen in a substance sample provided by the user and to generate allergen presence data, a processor operable to receive the IgA presence data from the biosensor and the allergen presence data from the allergen sensor, and a memory operable to store the IgA presence data and the allergen presence data.

### FIELD

The present disclosure relates generally to apparatuses, systems, and methods for measuring an inflammatory response. More specifically, the disclosure relates to apparatuses, systems, and methods determining the presence and cause of an inflammatory response.

### BACKGROUND

Allergic reactions can be the result of a variety of environmental factors. It is often difficult to determine which of multiple allergens may be responsible for an allergic reaction and even whether specific symptoms being experienced are in fact an allergic reaction.

One method for testing a person's allergic response to various environmental factors (e.g., substances) is the skin prick, puncture, or scratch test. A person is exposed to a panel of substances via a prick, puncture, or scratch on the persons epidermis. The location of the exposure is monitored to determine if an allergic response is occurring to the substance.

This common method of allergy testing is often effective. However, the method is administered by a medical professional, is limited to a predetermined panel of common allergens, does not address typical exposures in the course of a person's day, and does not provide feedback regarding potential exposures to allergens that may occur.

### SUMMARY

An allergic response and allergen detection system and method are provided for determining if an allergic response is occurring in an individual and if the substance to which the user is being exposed is a common allergen or an allergen to which the user is known to have an allergic reaction. The systems and methods described herein are operable to be implemented anywhere and at any time.

According to one example (“Example 1”), an allergy test kit is provided, the allergy test kit including a housing, a biosensor supported by the housing, the biosensor operable to detect a presence of immunoglobin A (IgA) in a sample of sweat of a user and to generate IgA presence data, an allergen sensor supported by the housing, the allergen sensor operable to detect a presence of an allergen in a substance sample provided by the user and to generate allergen presence data, a processor operable to receive the IgA presence data from the biosensor and the allergen presence data from the allergen sensor, and a memory operable to store the IgA presence data and the allergen presence data.

According to another example (“Example 2”), further to Example 1, the biosensor is operable to detect a concentration of IgA in the sample of sweat,

According to another example (“Example 3”), further to Example 1, the biosensor is operable to receive the sample of sweat from a swab used to collect the sample of sweat from the user.

According to another example (“Example 4”), further to Example 1, the biosensor is operable to be placed in direct contact with the user to receive the sample of sweat directly from the user.

According to another example (“Example 5”), further to Example 4, the housing is operable to be selectively worn by the user, wherein when the housing is worn by the user, the biosensor is positioned against a portion of an epidermis of the user.

According to another example (“Example 6”), further to Example 1, the allergy test kit further includes a removable insert supported by the housing proximate the allergen sensor, the removable insert operable to support the substance sample.

According to another example (“Example 7”), further to Example 1, the allergy test kit further includes a user interface, wherein the user interface is operably coupled to the processor and is operable to display at least one of whether an allergic reaction is detected and whether an allergen is detected.

According to another example (“Example 8”), further to Example 7, the user interface is supported by the housing.

According to another example (“Example 9”), further to Example 7, the allergy test kit further includes a wireless transceiver, wherein the user interface is operably coupled to the processor via the wireless transceiver.

According to another example (“Example 10”), further to Example 7, the memory stores a record of allergens designated by the user which trigger an allergic reaction in the user.

According to another example (“Example 11”), further to Example 10, the user interface notifies the user when the allergen sensor senses an allergen that is listed on the record of allergens designated by the user.

According to one example (“Example 12”), a system includes one or more processors of at least one computing device, and one or more memories with stored instructions that, when executed by at least one of the one or more processors, cause the at least one computing device to provide data representing at least one of an allergen and an allergic reaction, including recording detected allergen data received from and generated by an allergen sensor, the allergen sensor operable to detect an allergen in a sample provided to the allergen sensor, determining if at least a portion of the detected allergen data corresponds to at least a portion of known allergen data stored on the one or more memories, providing notification when at least the portion of the detected allergen data corresponds to at least the portion of the known allergen data, recording allergic response data received from and generated by a biosensor, the biosensor operable to detect IgA in a sample of sweat, determining if a threshold level of IgA is present in the sample of sweat, and providing notification when the threshold level of IgA is present in the sample of sweat.

According to another example (“Example 13”), further to Example 12, the one or more memories is rewritable to update the known allergen data to include new allergen data, and wherein the known allergen data may include a level of sensitivity.

According to another example (“Example 14”), further to Example 13, the stored instructions, when executed by the at least one or more processors, cause the at least one computing device to compare the detected allergen data to the known allergen data and the level of sensitivity, wherein the at least of the one or more processors provides notification if the detected allergen data is above a threshold of the level of sensitivity related to the known allergen data.

According to another example (“Example 15”), further to Example 12, the stored instructions, when executed by the at least one or more processors, cause the at least one computing device to sync data including allergen exposure data.

According to another example (“Example 16”), further to Example 15, the allergen exposure data is received from food logging and health databases, grocery order databases, restaurant order databases, food label databases, and geolocation databases.

According to another example (“Example 17”), further to Example 16, the allergen exposure data provided by input from a smart phone device that is carried by a user and that is one of the one or more computing devices.

According to another example (“Example 18”), further to Example 17, the smart phone includes a camera for capturing at least one of a receipt and an ingredient list, wherein the one or more processors determines exposure data based on the at least one of the receipt and the ingredient list.

According to another example (“Example 19”), further to Example 18, the stored instructions, when executed by the at least one or more processors, cause the at least one computing device to switch between an allergen module relating to the data representing allergens and an allergic reaction module relating to the data representing allergic reactions.

According to another example (“Example 20”), further to Example 12, the stored instructions, when executed by the at least one or more processors, cause the at least one computing device to timestamp detected allergen data and to timestamp the allergic response data.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a schematic of an allergy test kit for determining a presence and/or reaction to an allergen, in accordance with an embodiment.

FIG. 2 is an illustration of an embodiment of the allergy test kit of FIG. 1, including a housing and various components positioned on the housing and a user interface positioned separate from the housing, in accordance with an embodiment.

FIG. 3 is an illustration of an embodiment of the allergy test kit of FIG. 1, including a housing and various components positioned on the housing and a user interface positioned separate from the housing, the user interface including a processor and memory of the allergy test kit, in accordance with an embodiment.

FIG. 4 is an example method of implementing the allergy test kit, in accordance with an embodiment.

FIG. 5 is an example method of implementing the allergy test kit in conjunction with external sources for integrating data other than what is sensed directly by a biosensor and allergen sensor of the allergy test kit, in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

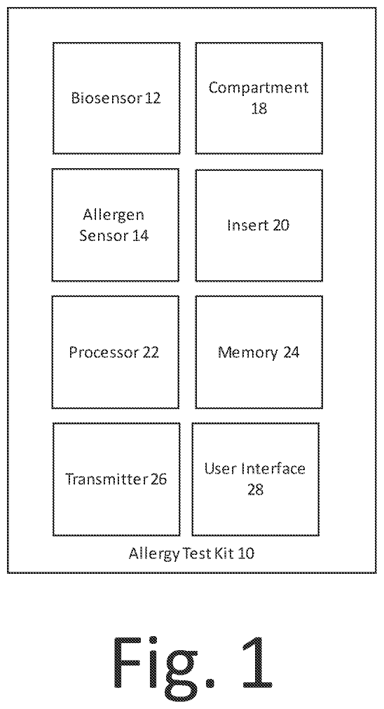

Referring to FIG. 1, schematic of an allergy test kit 10 is provided including a biosensor 12 and an allergen sensor 14, the allergy test kit 10 being operable to detect an allergic reaction in a user based on a sweat sample and to detect the presence of a known allergen in a substance sample (e.g., a food or chemical sample). The allergy test kit 10 can be used as a smart, personalized system operable to continuously track the user's data for potential allergens. The allergy test kit 10 can be used individually or can be used in conjunction with professional medical care, for example with a primary care physician, immunologist, allergist, rheumatologists, dietician, and so forth. By implementing the allergy test kit 10, the user is able to try new foods, eat available food, and avoid foods with increased confidence as well as track and understand the user's allergies (including food and non-food allergies) with more precision and confidence.

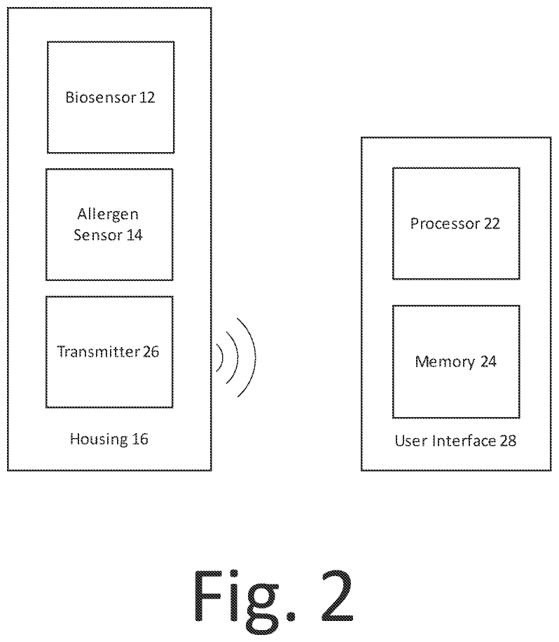

The biosensor 12 and the allergen sensor 14 are both supported by a single housing 16 (FIG. 2). The housing 16 is a handheld, portable housing. This allows the user to carry the allergy test kit 10 to the various places the allergy test kit 10 may be needed, including to a restaurant, school, camping, and so forth. This allows the user to pack the allergy test kit 10 in a backpack, handbag, and a console in a vehicle.

In some embodiments, the allergy test kit 10 may be synced or integrated with a mobile application that can be implemented on a smart phone and a tablet for added utility and functionality. The various functions and systems that can be implemented via a mobile application is described in further detail herein.

With further reference to FIG. 1, the biosensor 12 is provided and is operable to detect the presence of immunoglobin A (IgA) in a sample of sweat. The biosensor is supported by the housing 16 (FIG. 2). During an allergic response, IgA is secreted in the sweat of a user. Thus, in order to determine if an allergic response is occurring, the user is able to take a sample of sweat and have it analyzed by the biosensor 12. In some embodiments, the biosensor 12 is operable to determine levels or concentrations of IgA in a sample and can accordingly determine the level or severity of an allergic response. Thus, the user is able to determine not only if an allergic response is occurring, but how serious the response is. In some embodiments, the housing 16 includes a compartment 18 in which disposable swabs may be stored in order to collect a sample of sweat from the user. Once the disposable swab has collected the sweat sample, the disposable swab is presented to the biosensor 12 for the biosensor 12 to take a reading of the sweat sample. In other embodiments, the biosensor 12 is placed in direct contact with the user's skin/sweat in order to take the reading. Various biosensors 12 are known for detecting lgA (e.g., quartz crystal microbalance, field effect transistors, electrochemical impedance spectroscopy, interdigitated gold electrodes), including in the sweat of a user (e.g., field effect transistor biosensors), and therefore is not discussed in further detail herein.

The biosensor 12 is operable to generate lgA presence data relating to the reading or analysis of the sweat sample. The IgA presence data represents the presence of IgA detected or not detected in the sample of sweat of the user. In some embodiments, the IgA presence data indicates whether the presence of IgA in the sweat of the user is at or above a threshold amount or concentration in the user's sweat or a presence of IgA in an amount that is below the threshold amount or concentration (including a lack of IgA in the sweat). The threshold amount or concentration of IgA may be tuned (e.g., raised or lowered) as necessary to accurately provide data relating to an allergic response of the user (i.e., such as based on physical characteristics of the user). In some embodiments, the biosensor 12 is operable to detect a concentration of IgA in the sample of sweat and to generate IgA presence data including a concentration or levels detected by the biosensor 12. This allows the system not only to determine the presence of an allergic reaction in a user, but also the level or severity of the allergic reaction. The IgA presence data may be implemented in a variety of processes and may also be viewed by the user and/or health care professional as is discussed in further detail hereafter.

Referring still to FIG. 1, the allergen sensor 14 is operable to detect a presence of an allergen in a substance sample provided by the user. The allergen sensor is supported by the housing 16 (FIG. 2). Prior to, during, or after exposure to a substance, the user may implement the allergen sensor 14 to determine allergens that are present in a substance sample. For example, the user may take a substance sample from food that the user would like to consume. The allergen sensor 14 receives the substance sample and detects known or common allergens that are present in the substance sample (e.g., peanut oil, gluten, and so forth). The allergen sensor 14 may be operable to detect other allergens that are not from food samples, for example, allergens contained in other substances such as topical ointments, chemicals, and so forth. In some embodiments, the housing 16 includes an insert 20 into which the substance sample is placed or positioned for analysis. The insert 20 is positioned proximate the allergen sensor 14 such that when the insert 20 is positioned with the housing 16, the contents of the insert 20 are proximate or exposed to the allergen sensor 14. This allows the allergen sensor 14 to take a reading of the contents (e.g., the substance sample), to determine if there are any known or common allergens present in the substance sample. Various allergen sensors 14 are known for detecting known or common allergens in samples and therefore is not discussed in further detail herein.

The biosensor 12 is operable to generate allergen presence data relating to the reading or analysis of the substance sample. The allergen presence data represents the presence of allergens detected or not detected in the substance sample provided by the user. In some embodiments, the allergen presence data indicates whether the presence of allergens in the substance sample is at or above a threshold amount or concentration or the lack of allergens in an amount that is below the threshold amount or concentration. The threshold amount of detected allergen may be tuned (e.g., raised or lowered) as necessary to accurately provide data relating to the presence of an allergen. For example, the threshold amount may be automatically or manually lowered based on input relating to severe allergies of a user (e.g., peanut allergies). In some embodiments, the allergen presence data further includes concentration or levels detected by the allergen sensor 14. This allows the system not only to determine the presence of an allergen, but also the level or concentration of allergen in the substance sample. This may allow a user to determine if interacting with the sample might trigger an allergic response (e.g., a user with a gluten or lactose intolerance may see the levels of gluten or lactose in a food sample and may determine that the levels are not sufficient to illicit an allergic response or that prophylactic medication may be sufficient to limit allergic reaction post consumption). The allergen presence data may be implemented in a variety of processes and may also be viewed by the user and/or health care professional as is discussed in further detail hereafter.

Referring still to FIG. 1, the allergy test kit 10 includes a processor 22 which is operable to receive the IgA presence data from the biosensor 12 and the allergen presence data from the allergen sensor 14 and further includes a memory storage 24 that is operable to store the IgA presence data and the allergen presence data. The memory 24 may further include stored instructions that, when executed by the processor 22 provides the functionalities described herein of determining levels of an allergic reaction or levels of allergens in a substance sample, the stored instructions including instructions for reporting that information to the user and storing the information for use in other processes. The processor 22 and the memory 24 may both be supported by the housing 16 or separate from the housing 16, either individually or collectively. For example, the processor 22 and the memory 24 may be supported on a mobile device such as a smart phone. In such an embodiment, the sensors 12, 14 may communicate with the processor 22 and memory 24 of the mobile device via a transmitter.

For example, the allergy test kit 10 may include a transmitter 26 that is operable to transmit information to other components of the allergy test kit 10 (e.g., a monitor, computer, portable device, etc.) or to a device that is not part of the allergy test kit 10 with which the allergy test kit 10 interfaces (e.g., an existing computer or a cellular device via an application). The transmitter 26 may include a wire for a wired connection or the transmitter may be a wireless transmitter such as a transceiver. Various wireless protocols may be implemented as known in the field, including but not limited to near field communication, radio frequency, WiFi®, Bluetooth®, and so forth. The information or data transmitted via the transmitter 26 may include a variety of information. The information transmitted may vary depending on the configuration of the allergy test kit 10. For example, in some embodiments, the allergy test kit 10 may include a physical interface member 100 (see FIG. 2) that interfaces with the user and a computing member or a graphical user interface in order to compute and display the results of the reading. Any number of configurations may be implemented.

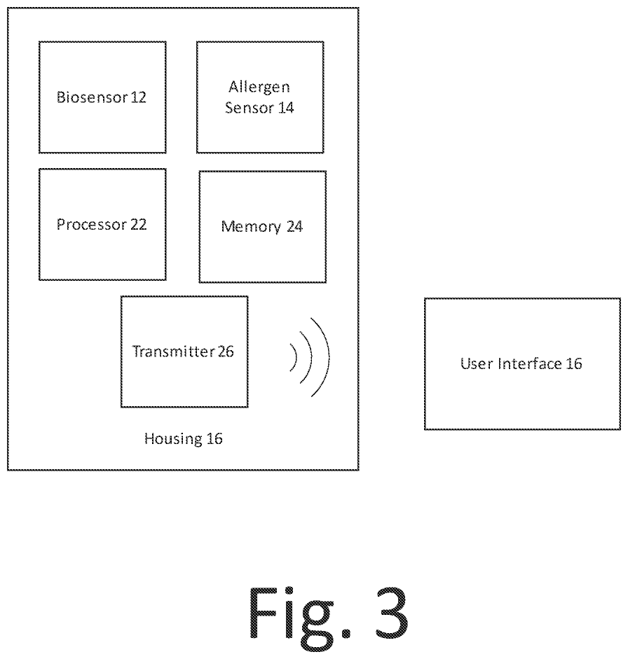

Referring to FIG. 2, an embodiment is illustrated including the housing 16 supporting the biosensor 12, the allergen sensor 14, and the transmitter 26. The housing portion of the allergy test kit 10 optionally include various portions of the allergy test kit 10 such as the processor 22 and the memory 24 (FIG. 3). A user interface 28 is provided and may be supported by the housing 16 or may be separate from the housing 16 (FIGS. 2 and 3). The user interface 28 allows the user to receive information from the allergy test kit 10, for example, a determination of an allergic response or the presence of an allergen may be alerted to a user via the user interface 28 (e.g., audible or visual alert). The user interface 28 may also be operable to provide information and/or instructions to the housing portion of the allergy test kit 10 and/or the processor 22. For example, with reference to FIG. 2, the user interface 28 may communicate with the biosensor 12 and allergen sensor 14 via the transmitter 26 to provide software or setting updates to the sensors 12, 14, including but not limited to threshold levels for detection, screening panels (e.g., allergens newly added to the panel for detection by the allergen sensor 14) . Referring to FIG. 3, the user interface 28 may communicate with the biosensor 12 and allergen sensor 14 via the transmitter 26 in order to update instructions, update the memory, provide software updates, and so forth.

Referring more specifically to the user interface 28, various embodiments of a user interface are contemplated herein. In one embodiment, the user interface includes a mobile device such as a smart phone. The mobile device may be programmed with an application that facilitates communication with the other components of the allergy test kit 10. The application allows the user to control the system such as initiating sensor activity as well as to view the data generated by the sensors 12, 14. For example, the application can provide the user with charts, tables, and visual representations of the data obtained relating to exposure to allergens and to allergic response by the user. The application can also provide timelines for exposure and allergic reactions based on time stamps assigned to the readings taken by the sensors 12, 14. This can allow the user and/or health care providers to better understand allergic responses of the user as well as what triggers may be causing the reactions. In some embodiments, the housing 16 can be worn by the user (e.g., a smart watch) such that the biosensor 12 is positioned against a portion of the epidermis of the user. This facilitates a real-time view over a period of time of an allergic response of the user. Thus, the application can show times at which a user may have demonstrated an allergic reaction, thus allowing the user to determine what exposures may have triggered the allergic reaction. The application also can provide notification to the user when the allergen sensor 14 senses an allergen that is listed on the record of allergens designated by the user or stored in the memory in the allergy test kit 10. The list of allergens may be manually selected by the user or health care professional (e.g., determined from a prior allergy screen or prick test) or may be automatically updated based on the data collected by the allergy test kit 10 (e.g., based on multiple allergic reactions over a period of time and as it relates to exposure to a specific allergen sensed by the allergen sensor 14).

In some embodiments, the allergy test kit 10 is operable to collect additional data relating to potential allergen exposure. For example, as previously described, the user may implement an application on a mobile device such as a smart phone (e.g., the user interface 28). The application may include location tracking over a period of time. The location of the user may be used to identify other allergens to which the user was exposed, for example, pollens and pollen levels based on the location data obtained by the mobile device. For example, the mobile device may provide a time and a location which can be used to determine that certain plants are blooming. In another example, the location and time data may be used to determine air quality which might cause an allergic response (e.g., inversions, smoke from wild fires, and so forth). Some or all of this information may be retrieved, including in real-time, from external sources, such as weather data sources or municipal data sources) In other embodiments, the location and time data may be used to provide data points at which the user has a specific allergic reaction which the user may use to manually determine an allergic reaction. For example, an allergic response is triggered due to the presence of a pet, but the data available is not sufficient to determine the presence of a pet. However, the allergy test kit 10 detects a repeated allergic response at a specific location and provides that information to the user. The user can then recognize the pattern and then assign meaning to the data point because the user knows that a pet is located at that location.

In some embodiments, the allergy test kit 10 is operable to obtain allergen data based on food labels. For example, the user interface 28 may include a camera that takes a photograph of a food label which is then scanned to determine which ingredients are present. The ingredients are saved in the memory 24 and provides further datapoints for determining exposures to allergens. The processor 22 is able to determine exposure data based on the captured image of the ingredient lists, receipts, and so forth. In some embodiments, the food labels may be manually entered. In some embodiments, OR or UPC codes may be scanned which includes the data relating to the ingredients. Similarly, the user may scan grocery receipts which can be used to determine exposures. When this type of data is captured by the allergy test kit 10 that may not indicate a specific time of exposure to an allergen (e.g., the food is prepared and consumed at a later time), this allergen data may be stored without a timestamp or with a timestamp including a range of time (e.g., a week). The allergy test kit 10 may then take the data over a period of time (e.g., a year) to identify patterns of allergic response based on the allergen data. Thus, the long-term data does not necessarily provide instantaneous feedback regarding an allergic response, but is used to determine an allergic response profile over time.

hi some embodiments, the allergy test kit 10 is operable to sync with other applications via a mobile device. For example, the user may use a food logging or health tracker application in the mobile device. The allergy test kit 10 can sync with the food logging and health tracker applications to determine which foods the user ingested. This prevents the redundant entry of data for food related allergen exposure by the user, Similarly, the allergy test kit may be operable to sync with applications for grocery ordering (e.g., grocery pickup or delivery applications). This data, similar to what was described previously, may be used to determine an allergic response profile over time based on the less specific exposure time that might be recorded by the allergy test kit 10. However, it is understood that the memory 24 may act as a repository, and the user and/or health care provider may manually parse through the information (e.g., food lists) to determine which potential allergens may be triggering an allergic reaction in the user.

In those embodiments in which a mobile device is implemented in connection with or supporting the allergy test kit 10, the mobile device may be implemented to track the user's location and store information such as which restaurants the user visited. This information can be stored and implemented for determining allergen exposure.

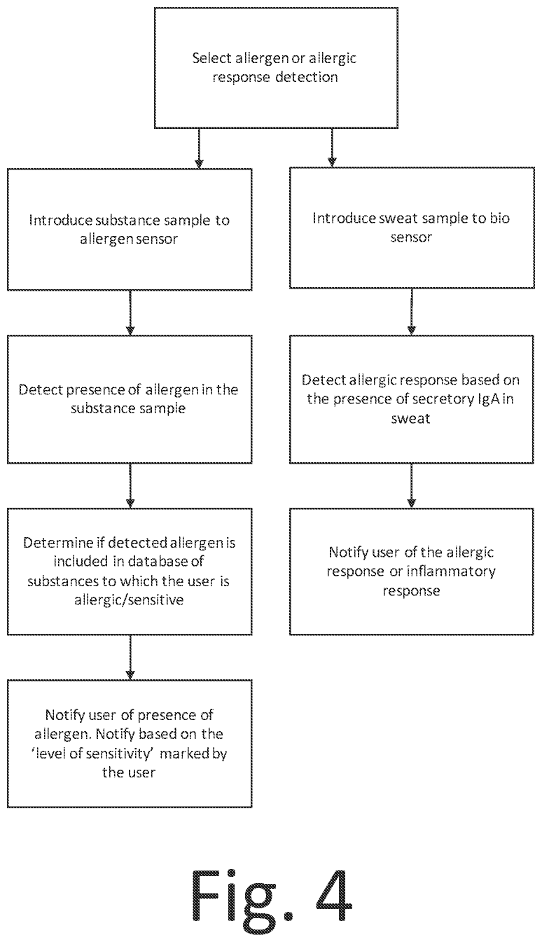

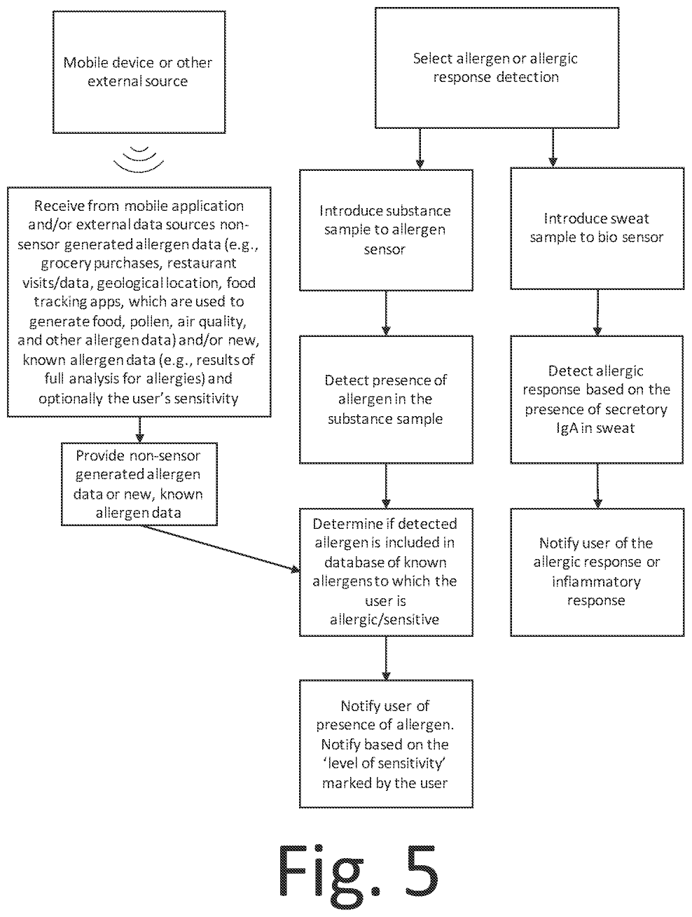

Referring to FIG. 4, the allergy test kit 10 can be implemented to determine exposure and/or allergic reaction to an allergen as described herein. The allergy test kit 10 can be switched between an exposure module and an allergic response module. The modules may be manually or may be automatically switched based on a variety of factors, for example which sensor 12, 14 is being engaged. Regarding the exposure module, the allergy test kit 10 is operable to detect an allergen in a sample provided to the allergen sensor 14 and to record detected allergen data generated by the allergen sensor 14. The allergen test kit 10 then determines if at least a portion of the detected allergen data corresponds to at least a portion of known allergen data stored on the memory 24. If the detected allergen data corresponds to at least a portion of known allergen data (for example, the allergen sensor detects peanut oil in a sample, and the system has been programmed to include peanuts as a known allergen), then the allergy test kit 10 provides notification of the correspondence (i.e., presence of a known allergen in the sample). In some embodiments, the allergy test kit 10 provides varying notification based on the known and detected allergens. For example, in embodiments implementing sensitivity levels, the allergy test kit 10 provides notification if the detected allergen data is above a threshold set for a specific known allergen. For example, a user may have a severe allergy that is triggered by minimal exposure and therefore the allergy test kit 10 will provide notification based on trace detection of the allergen. However, if the sensitivity level is of a lesser degree, the allergy test kit 10 provides notification to avoid a food when the amount or concentration of an allergen in a sample is above a predefined threshold based on the level of sensitivity to a specific allergen. As previously described, detected allergen data may also be based on information generated from sources other than the allergen sensor 14, for example, by manual entry of ingredients, scanned ingredient lists, scanned receipts, location data, and synced data from mobile applications (e.g., food logging and health databases, grocery order databases, restaurant order databases, food label databases, and geolocation databases) (FIG. 5).

The memory 24 of the allergy test kit 10 is rewritable in order to update the known allergen data to include new allergen data. In some embodiments the data recorded (i.e., known and new allergen data) may be assigned a sensitivity level based on the user's level of allergic response to the allergen, the sensitivity level also being modifiable based on further data or changes in levels of allergic response.

In the allergic response module, the allergy test kit 10 is operable to detect IgA in a sample of sweat via the biosensor 12 and to record allergic response data received from and generated by the biosensor 12. Thus the switch is between an allergen module relating to the data representing allergens and an allergic reaction module relating to the data representing allergic reactions. The allergy test kit 10 is operable to determine if a threshold level of IgA is present in the sample of sweat and to provide notification to the user when the threshold level of IgA is present in the sample of sweat. The allergy test kit 10 notifies the user of an allergic or inflammatory response when present. As previously discussed, the user may be notified in various ways, including visual and auditory notifications. In some embodiments, the allergy test kit 10 includes a color coded result for notifying the user of an allergic or inflammatory response. As previously discussed, external data may be integrated in such as external, lab-based allergy tests when further information is available. This can include levels of sensitivity to specific allergens. It is understood that lab-based allergy tests may be sought by the user because of the notifications provided by the allergy test kit 10 and may be implemented by a health care professional to perform a more targeted approach to the allergy test or panel.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. An allergy test kit comprising:
a housing;
a biosensor supported by the housing, the biosensor operable to detect a presence of immunoglobin A (IgA) in a sample of sweat of a user and to generate IgA presence data;
an allergen sensor supported by the housing, the allergen sensor operable to detect a presence of an allergen in a substance sample provided by the user and to generate allergen presence data;
a processor operable to receive the IgA presence data from the biosensor and the allergen presence data from the allergen sensor; and
a memory operable to store the IgA presence data and the allergen presence data.

2. The allergy test kit of claim 1, wherein the biosensor is operable to detect a concentration of IgA in the sample of sweat.

3. The allergy test kit of claim 1, wherein the biosensor is operable to receive the sample of sweat from a swab used to collect the sample of sweat from the user.

4. The allergy test kit of claim 1, wherein the biosensor is operable to be placed in direct contact with the user to receive the sample of sweat directly from the user.

5. The allergy test kit of claim 4, wherein the housing is operable to be selectively worn by the user, wherein when the housing is worn by the user, the biosensor is positioned against a portion of an epidermis of the user.

6. The allergy test kit of claim 1, further comprising a removable insert supported by the housing proximate the allergen sensor, the removable insert operable to support the substance sample.

7. The allergy test kit of claim 1, further comprising a user interface, wherein the user interface is operably coupled to the processor and is operable to display at least one of whether an allergic reaction is detected and whether an allergen is detected.

8. The allergy test kit of claim 7, wherein the user interface is supported by the housing.

9. The allergy test kit of claim 7, further comprising a wireless transceiver, wherein the user interface is operably coupled to the processor via the wireless transceiver.

10. The allergy test kit of claim 7, wherein the memory stores a record of allergens designated by the user which trigger an allergic reaction in the user.

11. The allergy test kit of claim 10, wherein the user interface notifies the user when the allergen sensor senses an allergen that is listed on the record of allergens designated by the user.

12. A system, comprising:
one or more processors of at least one computing device; and
one or more memories with stored instructions that, when executed by at least one of the one or more processors, cause the at least one computing device to provide data representing at least one of an allergen and an allergic reaction, including:
recording detected allergen data received from and generated by an allergen sensor, the allergen sensor operable to detect an allergen in a sample provided to the allergen sensor;
determining if at least a portion of the detected allergen data corresponds to at least a portion of known allergen data stored on the one or more memories;
providing notification when at least the portion of the detected allergen data corresponds to at least the portion of the known allergen data;
recording allergic response data received from and generated by a biosensor, the biosensor operable to detect IgA in a sample of sweat;
determining if a threshold level of IgA is present in the sample of sweat; and
providing notification when the threshold level of IgA is present in the sample of sweat.

13. The system of claim 12, wherein the one or more memories is rewritable to update the known allergen data to include new allergen data, and wherein the known allergen data may include a level of sensitivity.

14. The system of claim 13, wherein the stored instructions, when executed by the at least one or more processors, cause the at least one computing device to compare the detected allergen data to the known allergen data and the level of sensitivity, wherein the at least of the one or more processors provides notification if the detected allergen data is above a threshold of the level of sensitivity related to the known allergen data.

15. The system of claim 12, wherein the stored instructions, when executed by the at least one or more processors, cause the at least one computing device to sync data including allergen exposure data.

16. The system of cairn 15, wherein the allergen exposure data is received from food logging and health databases, grocery order databases, restaurant order databases, food label databases, and geolocation databases.

17. The system of claim 16, wherein the allergen exposure data provided by input from a smart phone device that is carried by a user and that is one of the one or more computing devices.

18. The system of claim 17, wherein the smart phone includes a camera for capturing at least one of a receipt and an ingredient list, wherein the one or more processors determines exposure data based on the at least one of the receipt and the ingredient list.

19. The system of claim 18, wherein the stored instructions, when executed by the at least one or more processors, cause the at least one computing device to switch between an allergen module relating to the data representing allergens and an allergic reaction module relating to the data representing allergic reactions.

20. The system of claim 12, wherein the stored instructions, when executed by the at least one or more processors, cause the at least one computing device to timestamp detected allergen data and to timestamp the allergic response data.
